(19) United States
(12) Patent Application Publication
(10) Pub. No.: US 2025/0305897 A1
(43) Pub. Date: Oct. 2, 2025

(54) PSEUDO SAMPLE FOR TEST METHOD VALIDATION

(71) Applicant: Regeneron Pharmaceuticals, Inc., Tarrytown, NY (US)

(72) Inventors: Connor Block, Somerville, MA (US); Joshua Kent, Rexford, NY (US)

(21) Appl. No.: 19/086,604

(22) Filed: Mar. 21, 2025

Related U.S. Application Data
(60) Provisional application No. 63/572,168, filed on Mar. 29, 2024.

(51) Int. Cl. G01L 5/00

(52) U.S. Cl. CPC G01L 5/0033

(57) ABSTRACT

Disclosed herein are a pseudo sample configured for validating the test method of measuring a force required to remove a cap from a drug delivery device, wherein the pseudo sample comprises an upper magnet movably secured within a body of the drug delivery device; and a cap magnet secured within the cap, wherein an attraction between the upper magnet and the cap magnet is configured to deliver a standard force required to remove the cap from the body of the drug delivery device. Also disclosed herein are methods of assembling such a pseudo sample, and methods of using such a pseudo sample to validate the test method of measuring a force required to remove a cap from a drug delivery device.

### CROSS REFERENCE TO RELATED APPLICATION

The present patent application claims priority under 35 U.S.C. § 119 (e) to U.S. Provisional Patent Application No. 63/572,168, filed Mar. 29, 2024, which application is incorporated by reference as though fully set forth herein.

### FIELD OF THE DISCLOSURE

Various embodiments of the disclosure relate to devices and methods adapted to validate test methods for drug delivery devices. In particular, embodiments of the disclosure relate to pseudo samples adapted for use in validating test methods used to test cap removal force for drug delivery devices, methods of assembling such pseudo samples, and methods of use of the same to perform test method validations as described herein.

### INTRODUCTION

Medical devices, including drug delivery devices, undergo testing and inspection throughout manufacture, including, e.g., incoming testing, in-process controls, and release testing, to verify that a subject device is free of defects and conforms to the requirements of its intended function. The set of procedures according to which the tests are conducted are known as test methods (TMs). The objectives of test methods may vary from test to test, and may include assessment of, e.g., whether a part is installed correctly, whether a design works over its expected input range, whether a calculation is performed correctly, whether any mechanical or design flaws are present, whether the device performs satisfactorily over varied periods of time and under stressed conditions, whether the device meets medical device cleanliness standards, and other objectives. Test methods may include direct measurement of parameters such as, e.g., voltage, distance, mass, force, durability, etc., or indirect measurement of parameters such as, e.g., a distance traveled, a time elapsed, distance over time, and so on.

Test method validation (TMV) is a documented process used to confirm through objective evidence that the test method used to perform a specific test, or to assess a particular parameter, is suitable for its intended purpose. Put another way, test method validation provides evidence that the testing method does not affect the measured result. TMV processes vary according to the test methods to be validated, and may include, for example, measurement system analysis (MSA) and other processes.

Validated methods provide confidence that the test method is appropriate and the data generated by the test method are reliable and repeatable. In turn, this contributes to the assurance of quality and safety of the medical device, and supports regulatory filings associated with such devices. Failure to properly validate test methods may result in clinical device failures, patient harm, and other unacceptable outcomes.

In the manufacture of certain medical devices such as, e.g., autoinjectors, prefilled syringes, and other drug delivery devices, test methods may be employed to test operational parameters such as cap removal force, activation force, and needle cover override force. In the example of cap removal force, this parameter is tested to ensure that the force required to remove the cap from the drug delivery device is neither too great nor too small. Where cap removal force, i.e. the amount of force required to remove the cap from the drug delivery device, is relatively greater, a user's ability to effectively de-cap the device may be decreased, and access to a medication may be undesirably slowed. Where cap removal force is relatively lesser, a user's ability to effectively de-cap the device may be increased, but other undesirable consequences may arise, such as increased incidence of accidental needle sticks prior to injection, premature expulsion of fluid (e.g., medication), and so on. Test methods may be employed to assess the cap removal force for a particular device, while test method validation establishes that the test method employed to test cap removal force is reliable, repeatable, and does not impact the device output for functional forces that are measured.

### SUMMARY

A first aspect of the disclosure provides a pseudo sample configured for validating a test method, the test method comprising measuring a force required to remove a cap from a drug delivery device, the pseudo sample comprising: an upper magnet adjustably fixed within a body of the drug delivery device; and a cap magnet secured within the cap, wherein an attraction between the upper magnet and the cap magnet is configured to deliver a standard force required to remove the cap from the body of the drug delivery device.

Various embodiments of the pseudo sample include one or more of the following features: an adjustable magnet assembly including the upper magnet, the adjustable magnet assembly being disposed within the body of the drug delivery device, and adapted to transport the upper magnet in a distal or proximal direction within the body of the drug delivery device, and to fix a position of the upper magnet after transport; and an upper magnet housing disposed about the upper magnet and within the body of the drug delivery device, wherein the adjustable magnet assembly is adapted to translate the upper magnet proximally or distally relative to the upper magnet housing. In various embodiments, the adjustable magnet assembly further comprises: a case at least partially enclosing the upper magnet, and including a stud extending proximally therefrom; a coupler sleeve disposed over a proximal end of the stud; and a rod having a distal end thereof disposed within the coupler sleeve, wherein the case, the coupler sleeve, and the rod are disposed within the upper magnet housing; the rod and the stud each comprise external threads, and the coupler sleeve comprises internal threads adapted to engage the external threads of the rod and stud.

Various embodiments of the pseudo sample include one or more of the following features: a heat-set threaded insert disposed about a portion of the rod that extends proximally beyond the coupler sleeve and within the upper magnet housing, wherein the heat-set threaded insert comprises internal threads adapted to engage the external threads of the rod; an opening extending through a thickness of a wall of the upper magnet housing and through a thickness of the heat-set threaded insert; and a fastener disposed in the opening, and adapted to maintain a position of the adjustable magnet assembly relative to the upper magnet housing. In various embodiments, the rod comprises steel.

Various embodiments of the pseudo sample include one or more of the following features: a bore extending through a full thickness of a wall of the body of the drug delivery device, and through a partial thickness of a wall of the upper magnet housing; and a pin disposed within the bore, thereby affixing an axial position and a rotational position of the upper magnet housing relative to the body of the drug delivery device. In various embodiments, the upper magnet housing comprises: a first outer diameter at a distal end thereof; and a second outer diameter at a proximal end thereof, the second outer diameter being smaller than the first outer diameter; and a shoulder disposed at a transition between the first and second outer diameters, wherein the shoulder is adapted to engage a feature on an inner surface of a wall of the body of the drug delivery device, thereby limiting proximal translation of the upper magnet housing relative to the body of the drug delivery device.

Various embodiments of the pseudo sample include one or more of the following features: a cap magnet housing disposed about the cap magnet and within the cap, wherein the cap magnet housing is axially affixed to each of the cap magnet and the cap. In various embodiments, one or more of the cap magnet or the upper magnet comprises neodymium, and one or more of the upper magnet housing or the cap magnet housing are formed by additive manufacturing. In various embodiments, the drug delivery device is an autoinjector or a pre-filled syringe, and the standard force is repeatable and reproducible.

A second aspect of the disclosure provides a method of assembling a pseudo sample configured for validating a test method, where the test method comprises measuring a force required to remove a cap from a drug delivery device. The method of assembling the pseudo sample comprises: providing a drug delivery device corresponding to the drug delivery device to be subjected to the test method; disassembling a portion of a body of the drug delivery device; disassembling a portion of a cap of the drug delivery device; assembling a first magnet into the cap; assembling a second magnet into the body, assembling the cap onto the body to form the pseudo sample, such that the second magnet is adapted to magnetically engage the first magnet; and confirming that the magnetic engagement of the first and second magnets generates a desired cap removal force.

Various embodiments of the method of assembling may additionally or alternatively include one or more of the following features: the second magnet is axially adjustable with respect to the body of the pseudo sample, wherein adjustment of the second magnet in a proximal direction reduces the cap removal force, and adjustment of the second magnet in a distal direction increases the cap removal force; and the desired cap removal force is a cap removal force equal to the cap removal force of the drug delivery device to be tested using the test method.

A third aspect of the disclosure provides a method for validating a test method, where the test method comprises measuring a force required to remove a cap from a drug delivery device. The method comprises: performing the test method on the drug delivery device; performing the test method on a pseudo sample as claimed in claim 1; designing a study to validate the test method, wherein the study includes a plurality of test runs; executing the study to validate the test method; and validating the test method upon obtaining results during the executing that meet pre-specified acceptance criteria. In various embodiments, the method further comprises: after performing the test method on the pseudo sample, adjusting the pseudo sample to increase or decrease the force required to remove the cap therefrom; and repeating the test method on the adjusted pseudo sample. In certain embodiments, performing the test method on the pseudo sample and adjusting the pseudo sample may be performed iteratively.

These and other aspects, advantages, and salient features of the disclosure will become apparent from the following detailed description, which, when taken in conjunction with the annexed drawings, disclose embodiments of the invention.

### BRIEF DESCRIPTION OF THE DRAWINGS

The accompanying drawings, which are incorporated into and constitute a part of this specification, illustrate various exemplary embodiments and, together with the description, serve to explain the principles of the disclosed embodiments. The drawings show different aspects of the present disclosure. Where appropriate, reference numerals illustrating like structures, components, materials, and/or elements in different figures are labeled similarly. It is understood that various combinations of the structures, components, and/or elements, other than those specifically shown, are contemplated and are within the scope of the present disclosure.

The embodiments described herein are not limited to any combinations and/or permutations of such aspects and/or embodiments. Moreover, each of the aspects of the described inventions, and/or embodiments thereof, may be employed alone or in combination with one or more of the other aspects of the described inventions and/or embodiments thereof. For the sake of brevity, certain permutations and combinations are not discussed and/or illustrated separately herein. Notably, an embodiment or implementation described herein as “exemplary” is not to be construed as preferred or advantageous, for example, over other embodiments or implementations; rather, it is intended reflect or indicate the embodiment(s) is/are “example” embodiment(s).

FIG. 1 shows a perspective view of a pseudo sample according to an embodiment of the disclosure.

FIG. 2 shows a cross sectional view of a pseudo sample according to an embodiment of the disclosure.

FIG. 3 shows a cross section of a perspective view of a pseudo sample according to an embodiment of the disclosure.

FIG. 4 shows a cross section taken along line A-A (FIG. 1) of a perspective view of a pseudo sample according to an embodiment of the disclosure.

FIG. 5 shows a cross section taken along line B-B (FIG. 3) of a perspective view of a pseudo sample according to an embodiment of the disclosure.

FIG. 6 shows a cross sectional view of a pseudo sample in which the cutting plane bisects the pseudo sample at the longitudinal axis thereof, according to an embodiment of the disclosure.

FIG. 7 shows a flow chart depicting steps in a method of assembling a pseudo sample according to an embodiment of the disclosure.

FIGS. 8-37 show perspective views of steps in the process of assembling a pseudo sample according to an embodiment of the disclosure.

FIG. 38 shows a flow chart depicting steps in a method of validating a test method using a pseudo sample according to an embodiment of the disclosure.

It is noted that the drawings of the disclosure are not necessarily to scale.

### DETAILED DESCRIPTION

Embodiments of the present disclosure relate to a pseudo sample configured for validating a test method, as well as methods of assembling the pseudo sample, and methods of performing test method validations using the pseudo sample, in which the test method to be validated is assessment of force required to remove a cap from a drug delivery device, i.e., the cap removal force.

As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” In addition, the terms “first,” “second,” and the like, do not denote any order, quantity, or importance, but rather are used herein to distinguish an element or a structure from another element or structure. Moreover, the terms “a” and “an” do not denote a limitation of quantity, but rather denote the presence of one or more of the referenced items.

The term “distal end” or any variation thereof, refers to the portion of a device farthest from an operator of the device, e.g., of a drug delivery device during an injection operation. For example, the distal end of a syringe would be the needle end of the syringe, and the distal end of a needle would be the sharp tip. Conversely, the term “proximal end,” or any variation thereof, refers to the portion of the device closest to the operator of the device during an injection operation. For example, the proximal end of a syringe would be the plunger end of the syringe, and the proximal end of a needle would be the end coupled to a needle hub. In the context of the pseudo sample described herein, these relative terms have their ordinary meanings regardless of the presence or absence of particular features such as, e.g., the needle. Further, as used herein, the terms “about,” “substantially,” and “approximately” generally mean ±10% of the indicated value. The terms “clinician” and “user” may be used interchangeably to refer to the individual performing methods as described herein.

### Pseudo Sample

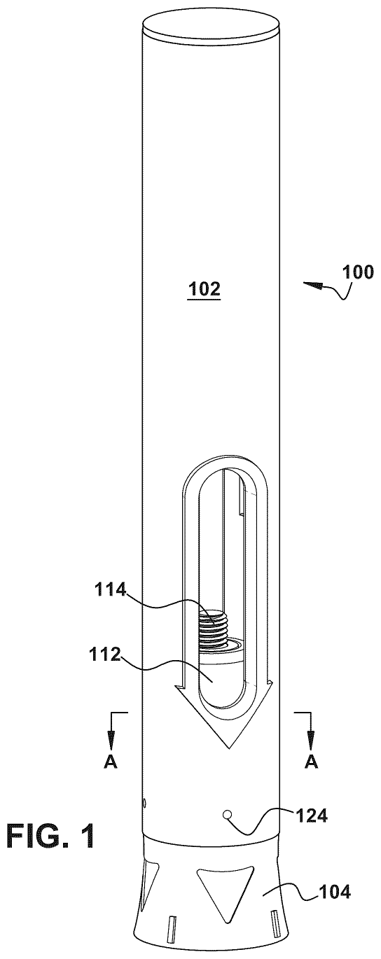

As indicated above, FIGS. 1-6 illustrate certain aspects of the disclosure that provide a pseudo sample 100 adapted to validate test methods for assessing cap removal forces on drug delivery devices. The pseudo sample 100 may be a modified version of the particular drug delivery device being tested, and for which the test method is being validated. The pseudo sample 100 may include magnetic material disposed within each of the cap as modified and the body as modified. The magnetic attraction between the respective cap and body magnets may be adapted to repeatably and reproducibly simulate a particular cap retention force. The cap removal force may be adjusted as described herein, by adjusting the distance separating the cap magnet and the upper magnet, located within the body of the device.

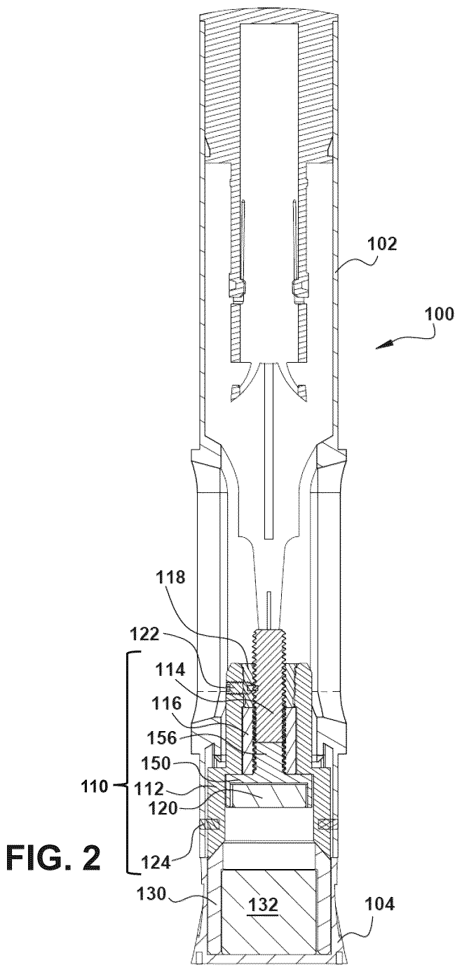

FIGS. 1 and 2 illustrate a pseudo sample 100 in perspective and cross sectional views, respectively. As shown, the pseudo sample 100 is configured for validating the test method of measuring a force required to remove a cap 104 from a body 102 of a drug delivery device. In certain embodiments, the drug delivery device may be, e.g., an autoinjector, a pre-filled syringe, or other drug delivery device. The pseudo sample 100 is adapted to provide a quantifiably standardized, repeatable, and reproducible force suitable to validate the test method as described herein.

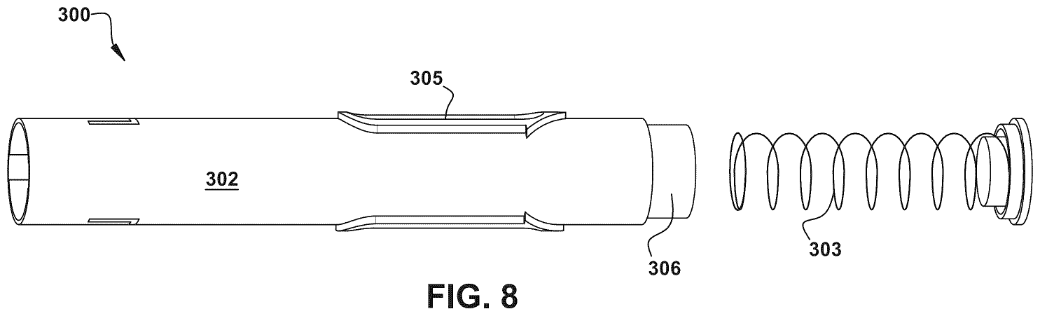

In certain embodiments, the pseudo sample may include a body 102 of the subject drug delivery device and the corresponding cap 104. The body 102 may be modified relative to a production sample 300 of the subject drug delivery device (FIGS. 8-10, 12-15) being tested, to remove certain internal components of the drug delivery device which do not affect the engagement or interaction of the body 102 with the cap 104.

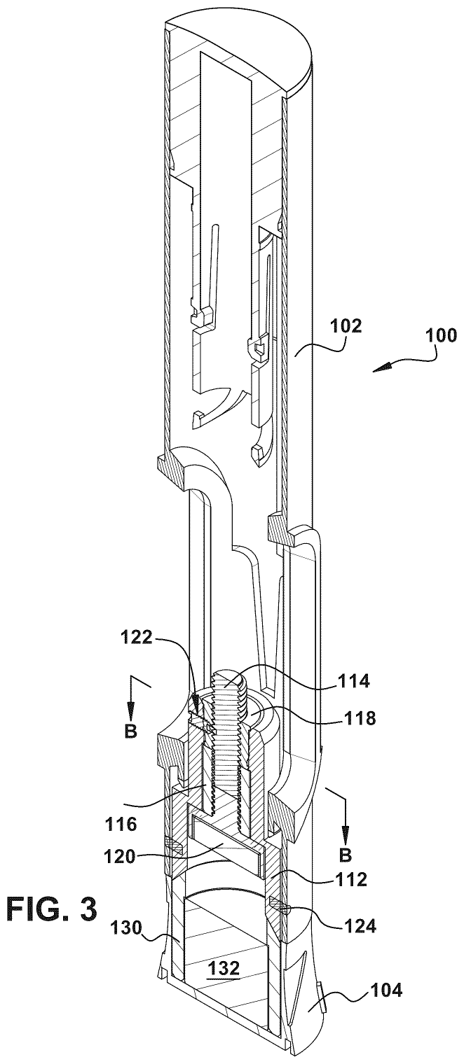

As shown in FIGS. 2-3, an upper magnet 120 may be movably secured within the body 102 by an adjustable magnet assembly 110. The adjustable magnet assembly 110 may include, for example, the upper magnet 120, an upper magnet housing 112, a coupler sleeve 116, and a rod 114, each of which are discussed further herein. The adjustable magnet assembly 110 may be adapted to translate the upper magnet 120 proximally or distally relative to, and within, the upper magnet housing 112 and the body 102 of the drug delivery device. In certain embodiments, the upper magnet 120 may be cylindrical in shape and may be, e.g., a neodymium magnet, which may be at least partially encased in a case 150 of, e.g., steel, with one of the north or south pole exposed. The upper magnet 120 may further include a threaded stud extending proximally therefrom.

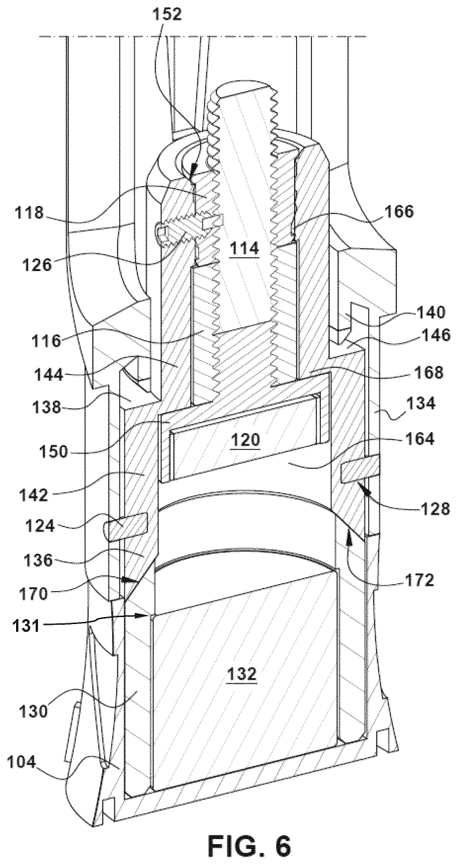

A cap magnet 132 may be secured within the cap 104, for example, by a cap magnet housing 130. The cap magnet housing 130 may partially encase the cap magnet 132, and may be disposed about and affixed to the cap magnet 132. In certain embodiments, the cap magnet 132 may be substantially annular in shape, and the cap magnet housing 130 may encase an outer circumferential surface of the cap magnet 132, while leaving a proximal-facing surface exposed or partially exposed. An adhesive such as, e.g., epoxy may be used to adhere the cap magnet 132 and a distal-facing surface of the cap magnet 132 to an interior proximal-facing surface of the cap magnet housing 130, leaving exposed at a proximal end a pole that is opposite the one of the north or south pole of the upper magnet 120 that is exposed, such that the distal end of the upper magnet 120 and the proximal end of the cap magnet 132 are adapted to attract one another with maximum pull. The cap magnet housing 130 may further be disposed within and affixed to the cap 104, such that rotation and translation are limited or prevented between the cap 104 and the cap magnet housing 130, and between the cap magnet housing 130 and the cap magnet 132. The cap magnet housing 130 may further include an internal shoulder 131, best shown in FIG. 6, disposed at or near a proximal end of the cap magnet housing 130. The shoulder 131 may include a distal-facing surface adapted to engage a circumferential edge of the proximal-facing surface of the cap magnet 132, and to resist translation of the cap magnet 132 out of the cap magnet housing 130 in the proximal direction under force.

The upper magnet 120 and the cap magnet 132 may be made of any known magnetic material, and the upper magnet 120 and cap magnet 132 may be made of the same or different materials as one another. In certain embodiments, one or both of the cap magnet 132 and/or upper magnet 120 may include neodymium. In further embodiments, one or more of the upper magnet housing 112 and the cap magnet housing 130 may be formed by additive manufacturing, and may be made of, e.g., thermoplastic materials. The upper magnet housing 112 and the cap magnet housing 130 may further include mating surfaces having complementary geometries to facilitate their engagement with one another. As shown in, e.g., FIGS. 2 and 6, the proximal end of the cap magnet housing 130 includes a frustoconical geometry which mates with a complementary geometry at the distal end of the upper magnet housing 112. The engagement of these features contributes to the centering of the cap 104 about the axis of the body 102.

When the body 102 and cap 104 are assembled, the magnetic attraction between the upper magnet 120 and the cap magnet 132 is configured to deliver a standard, repeatable, and reproducible amount of force. This amount of force represents the force required to de-cap the device, or to remove the cap 104 from the body 102 of the drug delivery device.

With reference to FIGS. 2, 3, and 6, as discussed above, the upper magnet housing 112 may be disposed about the upper magnet 120, and secured within the body 102 of the drug delivery device. The upper magnet housing 112 may be annular in shape, and may further include a hole or opening at a proximal end thereof, and a chamfer 152 (FIG. 6) disposed about an inner diameter of the hole. The upper magnet housing 112 may further include an outer diameter that varies along a longitudinal extent thereof, and an inner diameter that also varies along a longitudinal extent thereof. As shown in FIG. 6, the upper magnet housing 112 may have a first outer diameter 142 at a distal end thereof; and a second outer diameter 144 at a proximal end thereof. The second outer diameter 144 of the proximal end may be smaller than the first outer diameter 142 of the distal end, forming a shoulder 138 at the transition from the first outer diameter 142 to the second outer diameter 144. The shoulder 138 may include a proximal-facing surface 146 adapted to engage a feature 140 on an inner surface of a wall 134 of the body 102 of the drug delivery device, thereby limiting translation of the upper magnet housing 112 in the proximal direction relative to the body 102 of the drug delivery device. In this way, the shoulder 138 acts as a depth stop limiting the depth to which the upper magnet housing 112 may be inserted into the body 102. The axial length of the portion of the upper magnet housing 112 having the first outer diameter 142, i.e., the axial length of the portion distal of the shoulder 138, may be such that when the shoulder 138 contacts the feature 140, the distal end of the upper magnet housing 112 is approximately even with the distal end of the body 102. In certain embodiments, the proximal-facing surface 146 of shoulder 138 may be normal, substantially normal, or approximately normal to that of the second outer diameter 144.

The inner diameter of the upper magnet housing 112 may substantially follow the pattern described above with respect to the outer diameters 142, 144. In particular, the portion of the upper magnet housing 112 having the first outer diameter 142 may have a first inner diameter 164, and the portion of the upper magnet housing 112 having the second outer diameter 144 may have a second inner diameter 166 that is smaller than the first inner diameter 164. The inner diameter may also include an internal shoulder 168 at the transition from the first inner diameter 164 to the second inner diameter 166. This internal shoulder 168 may be adapted to act as a depth stop, limiting translation in the proximal direction of the upper magnet 120.

The upper magnet 120 may be substantially cylindrical in shape or disk-shaped, and may be at least partially encapsulated within a capsule or case 150. The case 150 may be disposed about an outer circumferential surface of the upper magnet 120 and a proximal-facing surface of the upper magnet 120, and may leave all or a portion of the distal-facing surface of the upper magnet 120 exposed. The exposed distal-facing surface of the upper magnet 120 may be adapted to magnetically engage the corresponding exposed proximal-facing surface of the cap magnet 132 as described herein. The case 150 may include a threaded stud disposed on a proximal-facing outer surface of the case 150, e.g., at a center thereof, and the threaded stud may extend longitudinally therefrom in a proximal direction.

The adjustable magnet assembly 110 may further include a coupler nut or sleeve 116 disposed over at least a portion of the stud 156, and extending in a proximal direction beyond the proximal end of the stud 156 itself, and a rod 114, a distal end of which may be disposed within the coupler sleeve 116, abutting a proximal end of the stud 156. The rod may be made of steel such as, e.g., grade B7 medium strength steel. The rod 114 and the stud 156 may each have external threads disposed thereon, and the coupler sleeve 116 may include a complementary internal thread pattern that is adapted to engage the external threads of the rod 114 and stud 156. A thread locking adhesive may further be used to maintain engagement of the external threads of rod 114 and stud 156 with the internal threads of coupler sleeve 116.

The rod 114 may be axially positioned such that a distal end thereof is disposed within the coupler sleeve 116, and a proximal end of the rod 114 may extend beyond the coupler sleeve 116 in the proximal direction. A substantially annularly shaped and threaded heat-set insert 118 may be disposed about the portion of the rod 114 that extends proximally beyond the coupler sleeve 116. The heat-set insert 118 may be, e.g., an M5 heat-set threaded insert, and may further include a geometry that is tapered at a distal end thereof. An inner diameter of the heat-set insert 118 may include internal threads adapted to engage the external threads on the rod 114, for example, including compatible thread count and pitch.

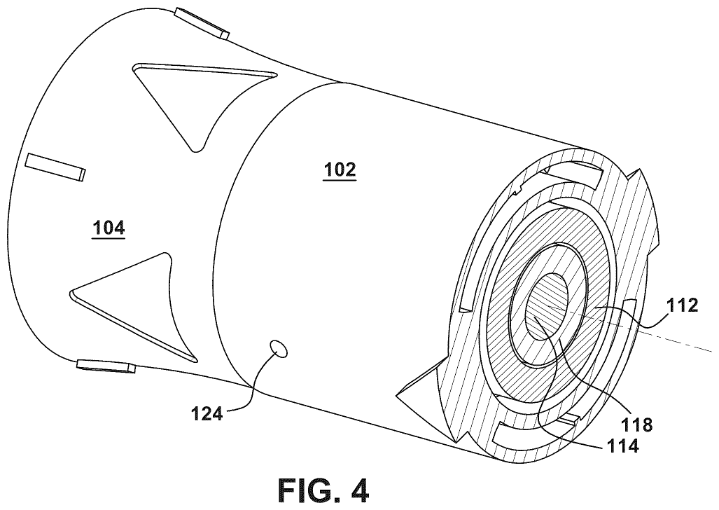

As shown in FIGS. 4 and 6, the upper magnet 120 encased within case 150, the coupler sleeve 116, the rod 114, and the heat-set insert 118 are all disposed within the upper magnet housing 112. The heat-set insert 118 may be disposed within the upper magnet housing 112 at a proximal end thereof, while the rod 114 may extend further in the proximal direction beyond the upper magnet housing 112 and the heat-set insert 118. Each of the upper magnet 120 encased within case 150, the coupler sleeve 116, the rod 114, the heat-set insert 118, the upper magnet housing 112, and the body 102 are maintained in a concentric arrangement by the close fit of the components.

As discussed above, the adjustable magnet assembly 110 facilitates the adjustment of the height of the upper magnet 120 relative to the upper magnet housing 112, and therefore relative to the body 102 of the drug delivery device and the cap magnet 132. Due to the threaded engagement of the encased upper magnet 120 via stud 156 and the coupler sleeve 116, the upper magnet 120 may be transported, or more particularly translated, in a distal direction by rotating the encased upper magnet 120 in a first direction, e.g. clockwise or counter clockwise, relative to the coupler sleeve 116. The upper magnet may similarly be transported, or more particularly translated in a proximal direction by rotating the encased upper magnet 120 including case 150 and stud 156 in a second direction, opposite the first, relative to the coupler sleeve 116. As the encased upper magnet 120 moves in a proximal direction, the distance between upper magnet 120 and the cap magnet 132 when assembled will increase, thereby reducing the force required to remove the cap 104 from the body 102. In contrast, as the encased upper magnet 120 moves in a distal direction, the upper magnet 120 and the cap magnet 132 will be closer together in the assembled pseudo sample 100, thereby increasing the amount of force required to remove the cap 104 from the body 102. In certain embodiments, the proximal opening in the cap magnet housing 130 has a circumference greater than that of the distal inner diameter 164 of the upper magnet housing 112. As a result, the diameter of the exposed proximal portion of the cap magnet 132 is greater than the diameter of the exposed distal portion of the upper magnet 120. This allows the upper and cap magnets 120, 132 respectively, to contact each other and provide the maximal cap removal force. In certain embodiments, the cap removal force may range from about 42 newtons when the upper magnet 120 and the cap magnet 132 are in contact with one another, to about 2 newtons when the upper magnet 120 and the cap magnet 132 are maximally separated, at 251 mm/min. In this manner, a user may adjust the removal force to a desired quantifiable amount.

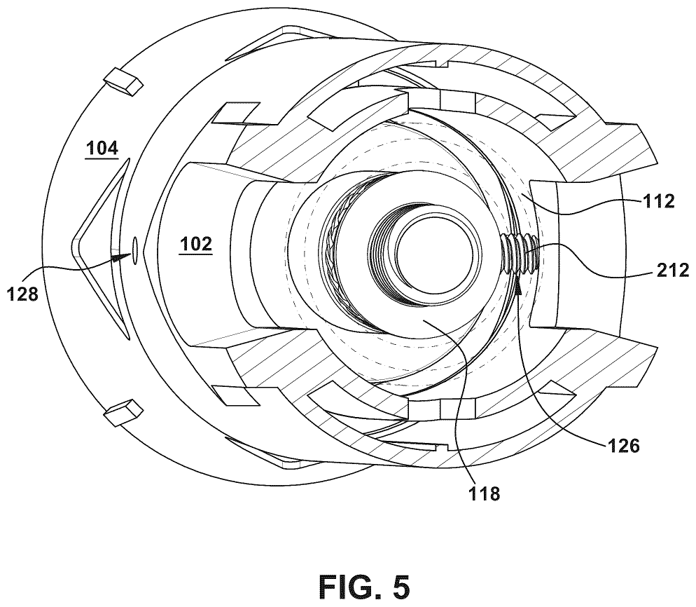

With reference to FIG. 5, a bore or opening 126 may extend through a thickness, e.g. a full thickness, of the wall 136 of the upper magnet housing 112, as well as through a thickness, e.g. a full thickness, of a wall of the heat-set insert 118, such that the opening 126 extends in a radially inward direction from an outer circumferential surface of the upper magnet housing 112 to the threads of the rod 114. Once the axial position of the upper magnet 120 is adjusted to an axial position corresponding to the desired amount of force required to remove the cap 104 from the body 102, a fastener 122 may be positioned in the opening 126. The fastener 122 contributes to the maintenance of the desired axial position of the adjustable magnet assembly 110, including the rod 114, coupler sleeve 116, heat-set insert 118, and upper magnet 120 relative to the upper magnet housing 112. In certain embodiments, the fastener 122 may include a set screw, and may particularly include a stainless steel, nylon-tipped set screw, and the opening 126 may include internal threads having a pitch and a thread count configured to threadably receive and engage the threads of the fastener 122, e.g., the set screw.

Where adjustments to cap removal force are desired after assembly of the pseudo sample 100 in whole or in part, the fastener 122 may be removed, the axial position of the upper magnet 120 may be adjusted relative to the upper magnet housing 112, and the fastener 122 may be replaced, thereby maintaining the adjusted position, and corresponding cap removal force. To facilitate such adjustments, the fastener 122 of the pseudo sample 100 may be accessed with a tool such as, e.g., a hex key 464, via a window opening such as window opening 305 (FIG. 8) in the body. Additionally, a screwdriver may be axially inserted into the body to rotate the threaded rod 114 to adjust the height of the upper magnet 120 relative to the body. Alternatively, the pseudo sample 100 may be disassembled in part or in whole to provide access, or to provide additional access to the fastener 122.

As shown in FIG. 6, a bore 128 may be disposed near a distal end of the body 102. The bore 128 may also extend through a full thickness of the wall 134 of the body 102 of the drug delivery device, and through at least a partial thickness of the wall 136 of the upper magnet housing 112. When the upper magnet housing 112 is properly positioned within the body 102, the bore 128 may extend radially inwardly from the outer circumferential surface of the wall 134 of body 102 and through a partial or full thickness of the upper magnet housing 112 wall 136. A pin 124 such as, e.g., a stainless steel dowel pin, may be positioned within the bore 128, thereby securing the axial position of the upper magnet housing 112 relative to the body 102 of the drug delivery device. As a result, the adjustment or maintenance of the position of the upper magnet 120 relative to the upper magnet housing 112 is equally adjusted or maintained relative to the body 102. In certain embodiments, one, two, three, or more bores 128 with corresponding pins 124 may be provided, which may be circumferentially spaced about the outer diameter of the body 102. In one embodiment, two bores 128 with corresponding pins 124 therein may be disposed about 180 degrees apart from one another around the outer circumference of the body 102. In another embodiment, one or more bore bores 128 having a pin 124 therein may be disposed along a mold seam line of the body 102.

As best seen in FIG. 6, the upper magnet housing 112 includes a chamfer 170 at a distal end thereof, while the cap magnet housing 130 includes a chamfer 172 at a proximal end thereof. These chamfers 170, 172 are complementary to one another, and facilitate mating engagement between the upper magnet housing 112 and the cap magnet housing 130 when the pseudo sample is assembled. The upper magnet housing 112 and cap magnet housing 130 are thus adapted to support and maintain the concentric engagement between the upper magnet 120 and cap magnet 132 within the assembled housings, and therefore, surface contact between upper magnet 120 and cap magnet 132 that is both ideal and controllable. In particular, the engagement between chamfers 170 and 172 limits or prevents translation of the cap magnet housing 130 relative to the upper magnet housing 112 along a plane perpendicular to the longitudinal axis of the pseudo sample.

### Methods of Assembling the Pseudo Sample

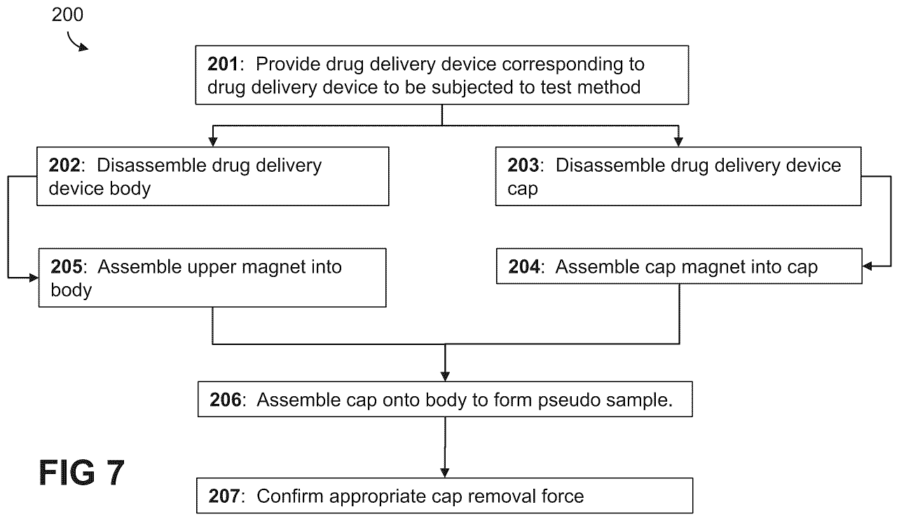

Turning next to the flow chart of FIG. 7 and the depictions of method steps in FIGS. 8-37, an exemplary method 200 of assembling a pseudo sample 100 (FIGS. 1-6) is described with reference to a flow chart depicting steps of the method as described herein (FIG. 7). FIGS. 8-37 provide illustrations of a device undergoing the steps of method 200 as described herein.

As noted, the steps of method 200 provide for the assembly of a pseudo sample 100, which may be a modified version of a production sample 300 of a drug delivery device. In particular, the test method may include testing, e.g., quantifying the cap removal force for removing the cap (not shown in FIG. 8) from the body 302 of the production sample 300. In certain embodiments, the production sample 300 of the drug delivery device may be an autoinjector. It is noted that the process illustrated in FIGS. 8-37 and described herein is only one example, illustrated using one drug delivery device production sample 300, e.g., one particular autoinjector. However, the assembly described herein may be adapted to assemble pseudo samples useful for test method validation in association with other drug delivery devices without departing from the scope of the invention. Methods of assembling a pseudo sample providing the same advantages and benefits as the one described herein are also considered to be part of the present disclosure. Such a drug delivery device and corresponding pseudo sample may include some or all of the particular features illustrated in FIGS. 8-37.

Turning to the method 200, shown in FIG. 7, in a first step 201, a production sample 300 of a drug delivery device is provided. The production sample 300 corresponds to the particular drug delivery device subject to the test methods and test method validations as described herein.

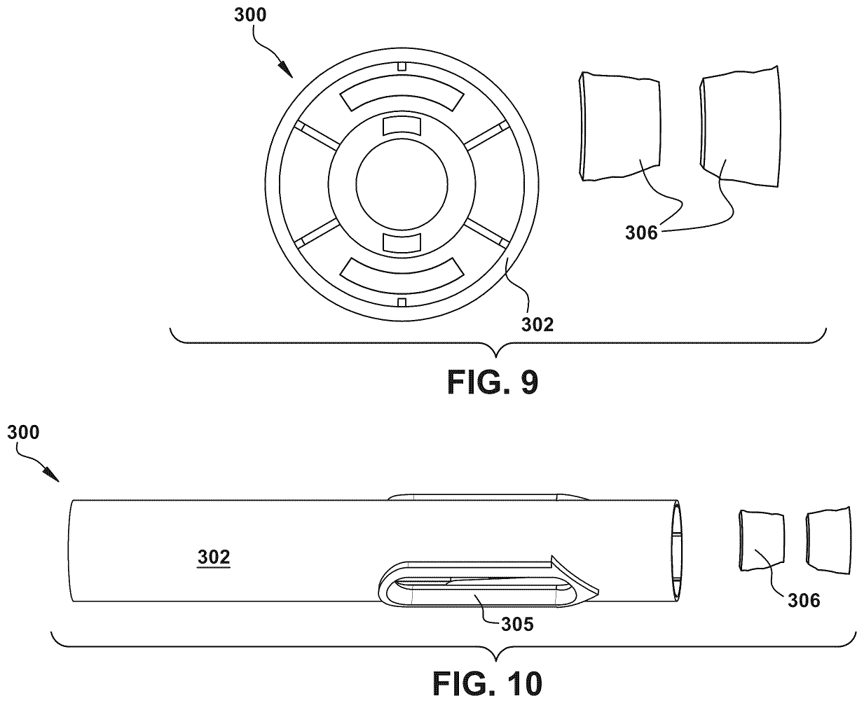

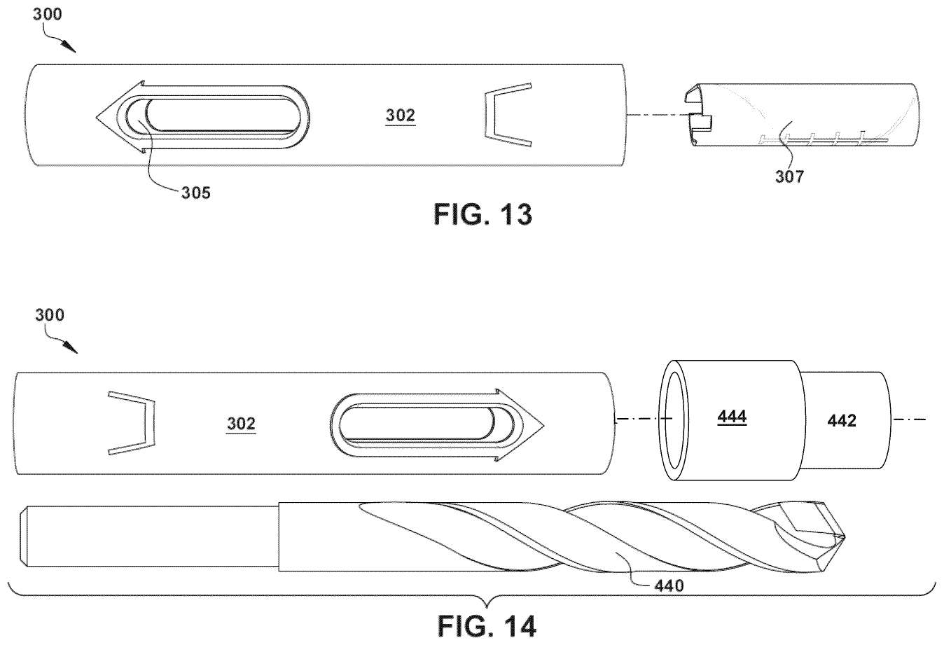

In step 202 (FIG. 7), the body 302 of the production sample 300 corresponding to the subject device may be at least partially disassembled. This may include, e.g., removal of at least a portion of the needle cover 306 (FIGS. 8-11) and syringe window 307 (FIG. 13) from the body 302 of the production sample 300. A cut may be made around the circumference of the base of the needle cover 306 (FIG. 8). An ultrasonic cutter such as, e.g., a WONDERCUTTER ultrasonic cutter (Cutra Co., Ltd., Incheon, Republic of Korea) may be used. A spring 303 may be removed from the front subassembly through the cut. Portions of the needle cover 306 that align with the window opening 305 in the production sample 300 may be removed (FIG. 9). With reference to FIG. 10, the protruding length of the needle cover 306 that extends past the distal end of the body 302 may also be removed, e.g., using the ultrasonic cutter.

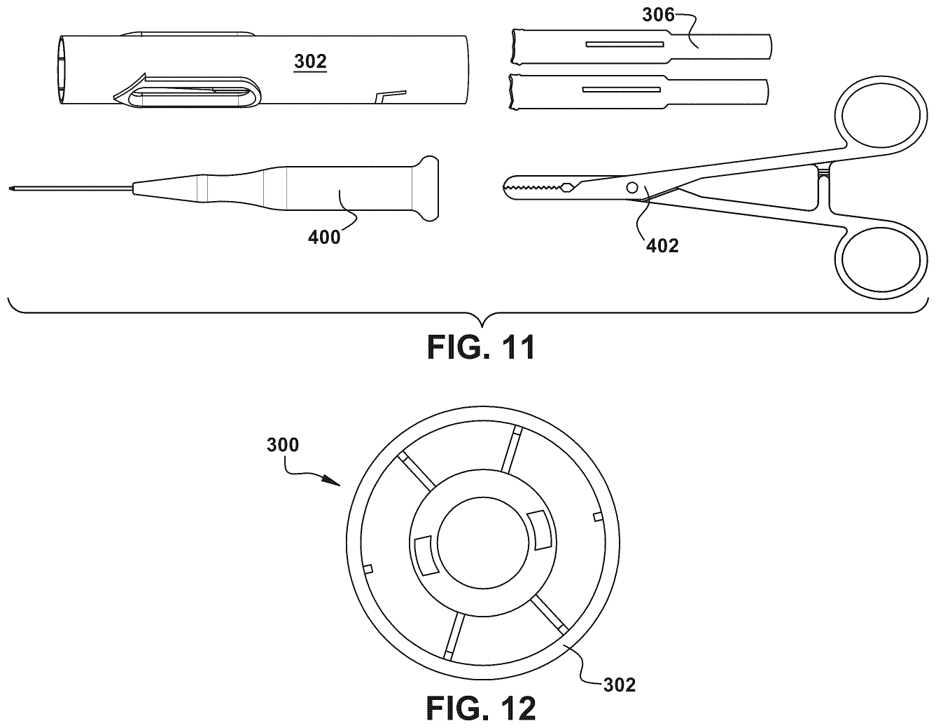

Referring to FIG. 11, the remaining pieces of the needle cover 306 may then be removed, e.g., using a flathead screwdriver 400 and forceps 402. This may include, for example, inserting the flathead screwdriver 400 through the proximal end of the body 302 of the production sample 300, and wedging the screwdriver 400 between the remaining portion of the needle cover 306 and the body 302. The flathead screwdriver 400 may be maintained in position with one hand, while the other hand may insert the forceps 402 into the proximal end of the body 302, and the user looks through the distal end of the body 302. The user may then take hold of the remaining portion of the needle cover 306 with the forceps 402 and lock the forceps 402. The flathead screwdriver 400 may then be removed from the body 302, and the remaining portion of the needle cover 306 may be pulled out of the proximal end of the body 302 using the forceps 402. This process may be repeated to remove remaining portion(s) of the needle cover 306 until the needle cover 306 is removed (FIG. 12).

Disassembly of the body 302 may further include cutting tabs, e.g. two tabs, that fasten the syringe window 307 into the body 302 (FIG. 13). This may be performed by inserting the ultrasonic cutter into the distal end of the body 302 and cutting the tab(s). The syringe window 307 may then be removed from the body 302 by pulling the syringe window 307 out of the proximal end of the body 302.

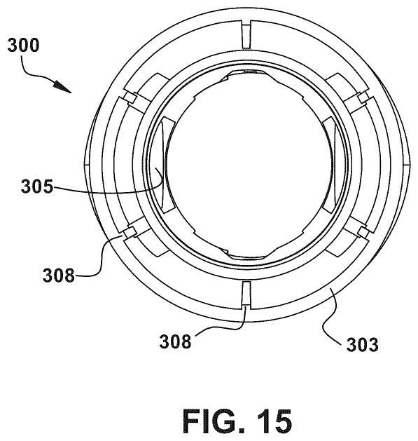

Referring to FIGS. 14-15, internal material may be removed from the body 302 of the production sample 300. This may be accomplished by inserting the proximal end of a drill bushing jig 444 onto or over the distal end of the body 302. The drill bushing jig 444 may include internal features at the proximal end thereof (not shown) which are adapted to engage the ribs 308 (FIG. 15) on the interior of the body 302, e.g., in a complementarily shaped manner, to properly center and axially align the drill bushing 444 relative to the body 302. The drill bushing jig 444 may be, e.g., formed by additive manufacturing (3D printing), and may further include a lip at the proximal end that is adapted to slip over the distal end of the body 302. A drill bushing 442 may be coupled to the drill bushing jig 444, e.g., by pressing the proximal end of the drill bushing 442 into the distal end of the drill bushing jig 444. A friction fit may be provided between the drill bushing 442 and the jig 444. A drill bit 440 corresponding to the dimensions of the drill bushing 442, e.g. a 15 mm drill bit, may then be inserted into the drill bushing 442 by hand. The drill bit 440 may be used, either by hand or with a drill, to remove internal material within the body 302 below, or distal relative to the window opening 305 (FIG. 15). Any excess material may be removed, e.g., with a precision cutting knife (e.g., an X-ACTO knife, Elmer's Products Inc., Westerville, OH) and/or forceps 402 (FIG. 11).

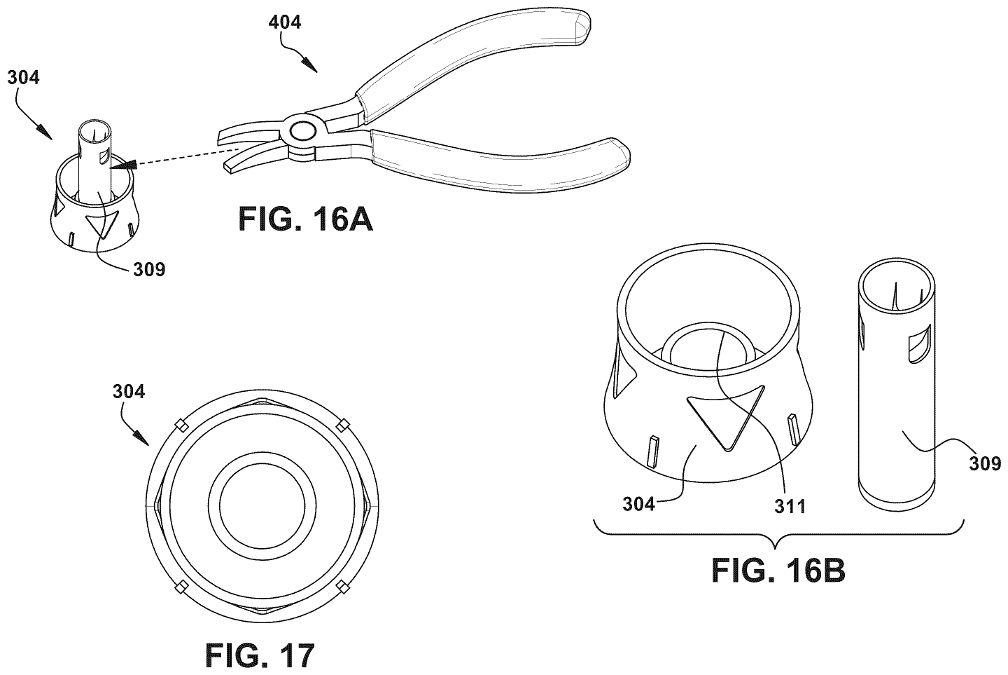

In step 203 (FIG. 7), the cap 304 of the production sample 300 corresponding to the subject device may be at least partially disassembled. In various embodiments, step 203 may occur either before, contemporaneously with, or after steps 202 (discussed above) and 205 (discussed below). Disassembly of the cap 304 may include, e.g., removal of a soft needle shield receiver 309 and a soft needle shield receiver holder 311 from an interior of the cap 304 (shown in FIGS. 16A-16B). The soft needle shield receiver 309, which may be embedded into the cap 304, may be removed therefrom, e.g., by using a pair of needle nose pliers 404 to twist the soft needle shield receiver 309 to break the adhesive affixing it to the cap 304. The soft needle shield receiver 309 may then be removed from the cap 304 by pulling upward and away from the cap 304 with the needle nose pliers 404. Referring to FIG. 17, the soft needle shield receiver holder 311 (FIG. 16B) may also be removed. This may be performed using a drill press and an endmill, e.g. a 0.5 in (12.7 mm) endmill, to remove the soft needle shield receiver holder 311 from the proximal-facing inner surface at the distal end of the cap 304. Sub-steps of the removal may include placing machinist parallel bars inside of the vise on the drill press, and placing the cap 304 onto the machinist parallel bars and securing it using the vise, being careful not to permanently deform the cap 304 when tightening the vise. The removal may further include inserting the endmill into the drill press chuck and tightening the chuck in place using a chuck key, adjusting a height of the drill press bed and the alignment of the vise so the endmill can completely remove the soft needle shield receiver holder 311, and pushing down on the drill press crank handle and removing the soft needle shield receiver holder 311. Downward motion may be continued until a change in the material is heard and/or felt.

Once disassembled according to step 203, the cap 304 of the production sample 300 may be modified to form the cap 104 of the pseudo sample 100 (FIGS. 1-3) in step 204 of the method 200 (FIG. 7). Step 204 may be performed following step 203, but may occur before, contemporaneously with, or after steps 202 and 205.

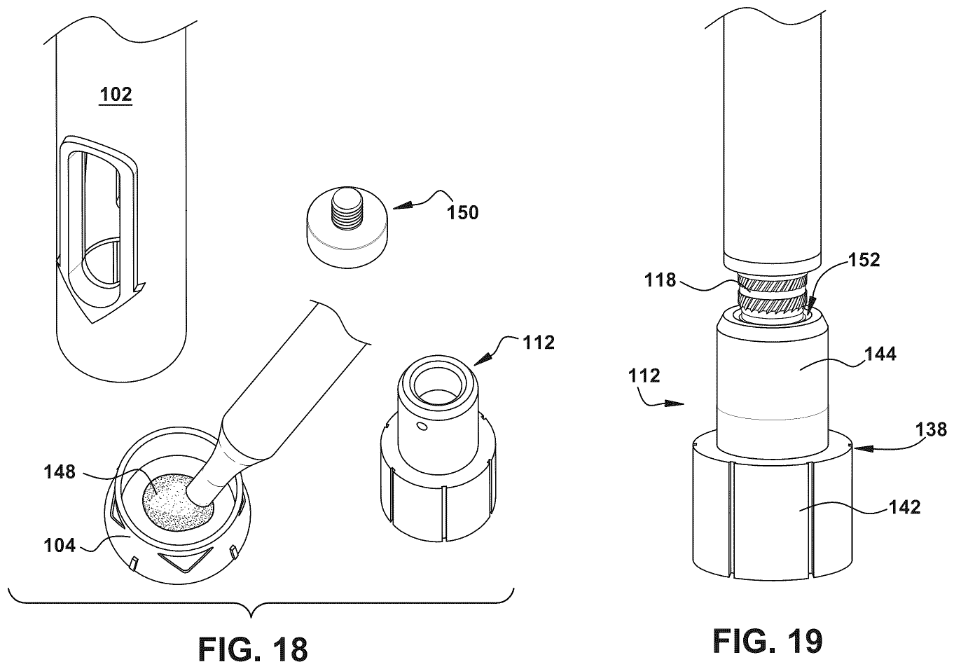

In step 204, a cap magnet housing 130 may be provided. The cap magnet housing 130 may be formed by, e.g., additive manufacturing/3D printing, and may be made of, e.g., a thermoplastic material. The cap magnet housing 130 may be hand filed as or if needed to remove any remnants from the 3D printer. The cap magnet 132 may be test fitted into the substantially annular cap magnet housing 130 inside of cap 104, and checked to confirm that the magnetic poles align properly with the encapsulated upper magnet 120 (discussed further above and below). An adhesive 148, e.g. epoxy, may be used to affix a distal surface of each of the cap magnet 132 and cap magnet housing 130 into the cap 104. The adhesive 148 may be applied by installing the epoxy and fine tip epoxy nozzle into an epoxy gun, expelling epoxy from the gun to ensure the fine tip is working properly. The cap magnet 132 and cap magnet housing 130 may be removed from the cap 104 after test fitting, making sure not to change the orientation of the cap magnet 132. A thin layer of adhesive 148 may be applied into the proximal facing inner surface at the distal end or bottom of the cap 104 (FIG. 18). The cap magnet 132 and cap magnet housing 130 may then be pressed down into the epoxy, such that both of the cap magnet 132 and the cap magnet housing 130 adhere directly to the cap 104. The cap magnet 132 is now assembled into the cap 104.

In step 205 of the method of FIG. 7, an upper magnet 120 is assembled into the body 102 of the pseudo sample. As shown in FIG. 19, an upper magnet housing 112 is provided, having an annular shape, and an opening extending longitudinally therethrough. A chamfer 152 may be disposed about an inner diameter of the annular upper magnet housing at a proximal end thereof. The upper magnet housing 112 may further include an outer diameter that varies along a longitudinal extent thereof, including a first outer diameter 142 at a distal end thereof; and a second, smaller outer diameter 144 at a proximal end thereof. A shoulder 138 may be disposed at the transition from the first outer diameter 142 to the second outer diameter 144.

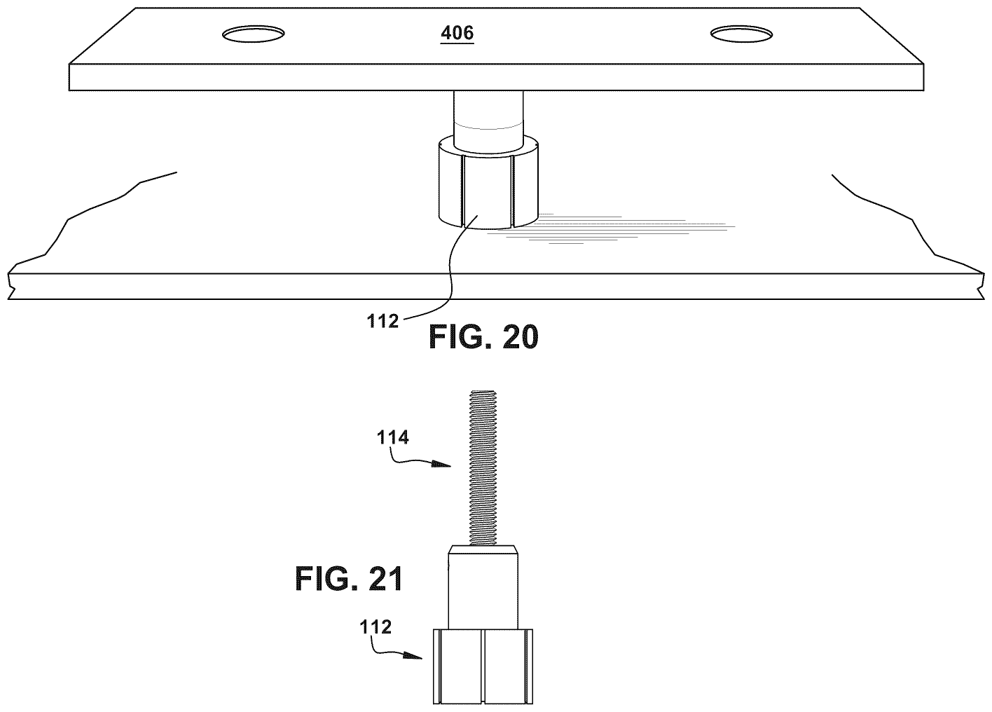

A heat-set insert 118 such as, e.g., a tapered M5 heat-set insert, may be installed into the substantially annular shaped proximal end of the upper magnet housing 112. This may include inserting a heat-set insert tip corresponding to the particular heat-set insert 118, e.g. M5 heat-set insert tip, into a soldering iron and allowing it to heat up. The heat-set insert 118 may then be placed into the opening at the proximal end of the upper magnet housing 112, which may include a chamfer 152 disposed about an inner diameter of the opening. The soldering iron tip may be inserted into the heat-set insert 118, and the heat-set insert 118 may be pressed down into the upper magnet housing 112 until the heat-set insert 118 is substantially inserted into the upper magnet housing 112, e.g., such that only approximately 1 mm of the heat-set insert 118 protrudes from the proximal end of the upper magnet housing 112. Referring to FIG. 20, the heat-set insert 118 may then be pressed into the upper magnet housing 112 by placing a machinist parallel bar 406 on top of the heat-set insert 118 and pressing it down until the machinist parallel bar 406 is sitting on top of the upper magnet housing 112. The heat-set insert 118 may then be allowed to cool with the machinist parallel bar 406 resting thereon, e.g., for at least a minute, before removing the upper magnet housing 112 and heat-set insert 118. As shown in FIG. 21, a rod 114 may then be threaded through the heat-set insert 118 to clear any melted plastic away from the internal threads.

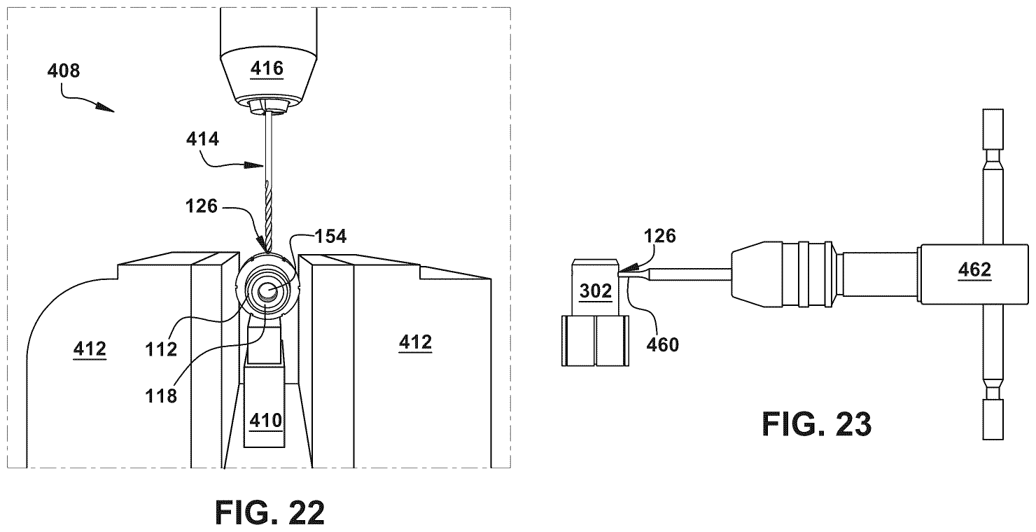

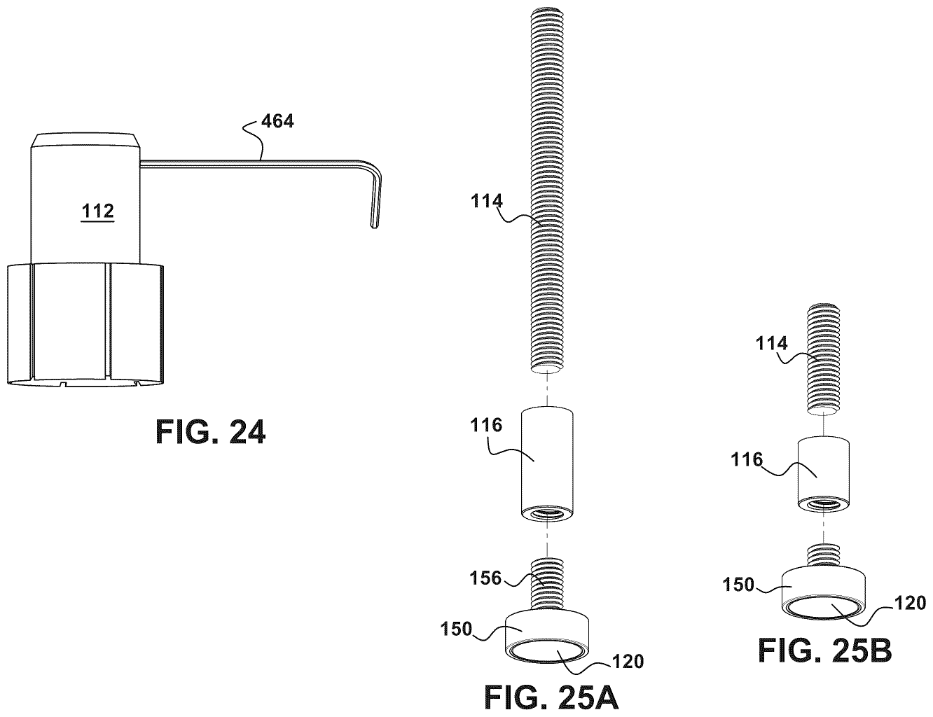

As shown in FIG. 22, a hole or opening 126 may be formed, e.g. drilled, in the upper magnet housing 112 in a radially inward direction from the outer diameter thereof. The opening 126 may be perpendicular or substantially perpendicular to a longitudinal axis of the upper magnet housing 112. The opening 126 may have a diameter of, e.g., about 1.6 mm, and may be adapted to receive a fastener 122 such as, e.g., an 18-8 stainless steel nylon-tip set screw. The drilling of opening 126 may be performed using a precision drill press 408. This may include placing metal spacer blocks 410 inside of the precision drill press vise 412, inserting a drill bit 414 corresponding to the desired size of opening 126, e.g. 1.6 mm, into the precision drill press chuck 416, and tightening the drill bit 414 into place using the chuck key. The upper magnet housing 112 may be placed horizontally on the metal spacer blocks 410, with the second, smaller outer diameter 144 at the proximal end (FIG. 19) of the upper magnet housing 112 resting on the metal spacer blocks 410, and the vise 412 may be tightened. The pre-existing longitudinally extending opening 154 in the heat-set insert 118 may be vertically aligned with the drill bit as shown in FIG. 22. The precision drill press 408 crank handle may be pushed down until the drill bit 414 passes through the outer face of the heat-set insert 118, through a full thickness of the wall of the heat-set insert 118, and into the center of the heat-set insert 118. The upper magnet housing 112 may then be removed from the precision drill press 408. As shown in FIG. 23, a tap 460, e.g. an M2 tap, may be inserted into a T-handle tap wrench 462. The hole or opening 126 in the upper magnet housing 112 and heat-set insert 118 may then be threaded by inserting the tap 460 into the opening 126 and twisting the T-handle tap wrench 462 clockwise, while applying pressure into the opening 126. A fastener 122 may then be inserted into the opening 126. This may include, e.g., inserting a hex key 464 such as a 0.9 mm hex key into a set screw serving as a fastener 122 as shown in FIG. 24, and turning the hex key 464 clockwise to thread the set screw into the threaded opening 126 of upper magnet housing 112. The fastener 122 may be inserted into the opening 126 until the fastener 122 does not protrude outside of the outer circumferential surface of the upper magnet housing 112.

Turning to FIGS. 25A-25B, one or more of the stud 156 on encapsulated upper magnet 120, the coupler sleeve 116, or the rod 114 may be cut down if a shorter length is desired, e.g., from their uncut dimensions (FIG. 25A) to their cut dimensions (FIG. 25B). These cuts may be made by putting each component in a vise and cutting to a desired length with an angle grinder. In certain embodiments, the stud 156 of the encapsulated upper magnet 120 may be cut down to about 5 mm, the coupler sleeve 116 to about 10 mm, and the rod 114 to about 18 mm. A slit 162 may then be cut into a proximal end of the rod 114 (best seen in FIG. 30B). A rotary tool such as, e.g., a DREMEL® rotary tool (Robert Bosch Tool Corporation) with a thin-cut abrasive cutoff wheel may be used to cut slit 162.

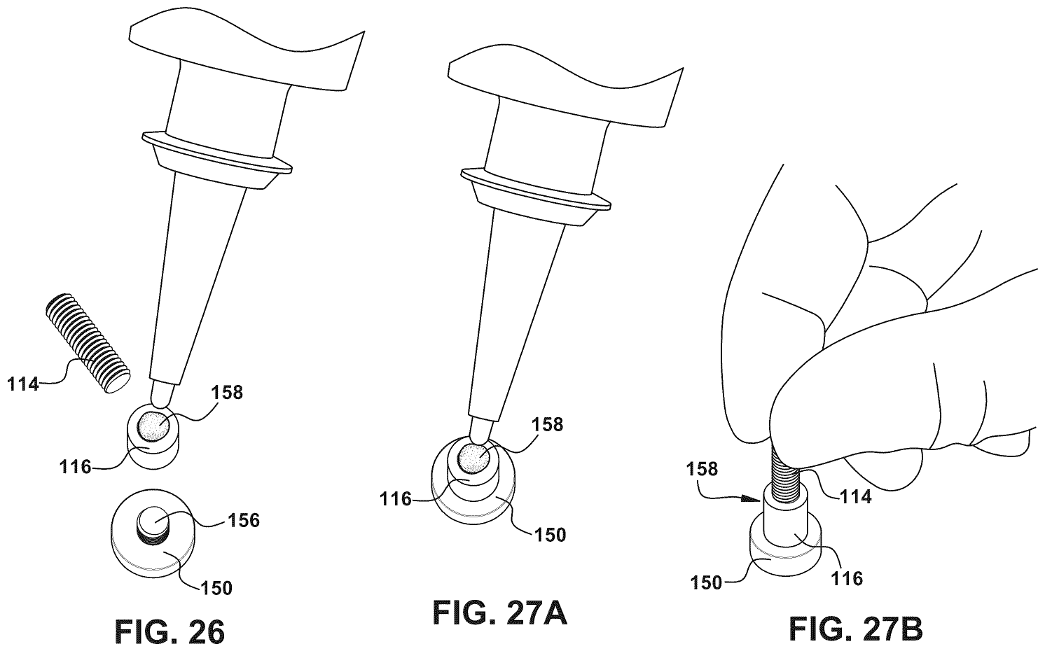

As shown in FIGS. 26, 27A, and 27B, an adhesive 158 such as, e.g., LOCTITE Threadlocker (Henkel IP & Holding, GmbH) may be used to seal the upper magnet 120 case 150 with threaded stud 156, the coupler sleeve 116, and the rod 114 together to form part of the adjustable magnet assembly 110. As shown in FIG. 27, a bead of adhesive 158 may be applied to the inner diameter on one end, e.g., the distal end of the coupler sleeve 116, and the threaded stud 156 of case 150 surrounding upper magnet 120 may be threaded into the bead of adhesive. As shown in FIG. 27A, a second bead of adhesive 158 may be applied to the inner diameter of the other end, e.g. the proximal end of the coupler sleeve 116, and the rod 114 may be threaded into the bead of adhesive 158 to install the rod 114 (FIG. 27B). Any excess adhesive 158 may then be wiped away from the assembled portion of the adjustable magnet assembly 110, and the adhesive 158 may be allowed to dry.

The portion of the adjustable magnet assembly 110 may then be threaded into the upper magnet housing 112 to assist in the installation of the upper magnet housing 112 into the body 102. The fastener 122 may be removed from the upper magnet housing 112 and heat-set insert 118, e.g., where the fastener 122 is a set screw, by using the hex key 464 to back the set screw out by rotating the hex key 464 in a first direction (e.g., counterclockwise). The adjustable magnet assembly 110 may then be threaded into the upper magnet housing 112 by rotating the adjustable magnet assembly 110 clockwise relative to the heat-set insert 118. The internal threads of the heat-set insert 118 are adapted to threadably engage the external threads of rod 114, causing the rod 114 to translate together with the coupler sleeve 116 and the upper magnet 120 encapsulated within case 150 as the rod is rotated within the heat-set insert 118. When the desired axial position of the upper magnet 120 is achieved relative to the upper magnet housing 112, the fastener 122 may then be re-installed into opening 126 in the upper magnet housing 112 and heat-set insert 118. For example, where the fastener 122 is a set screw, this may be accomplished by inserting the set screw into the opening 126 and rotating the hex key 464 in a second direction opposite the first direction (e.g., clockwise).

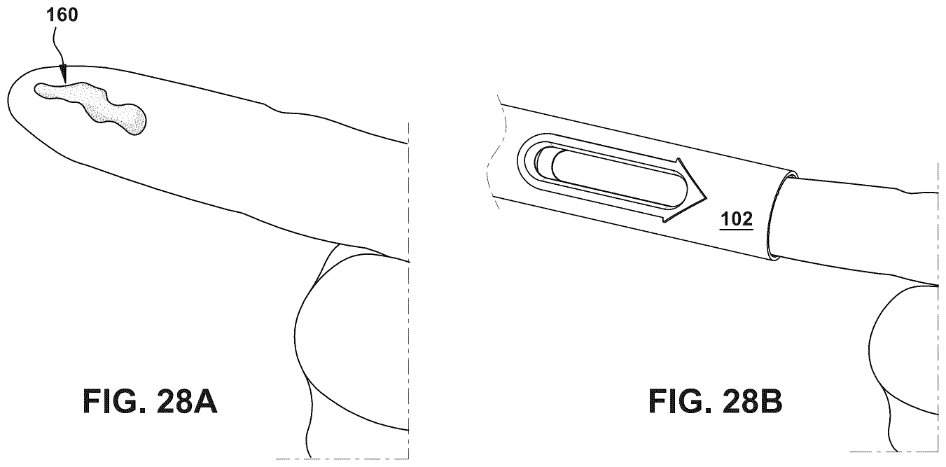

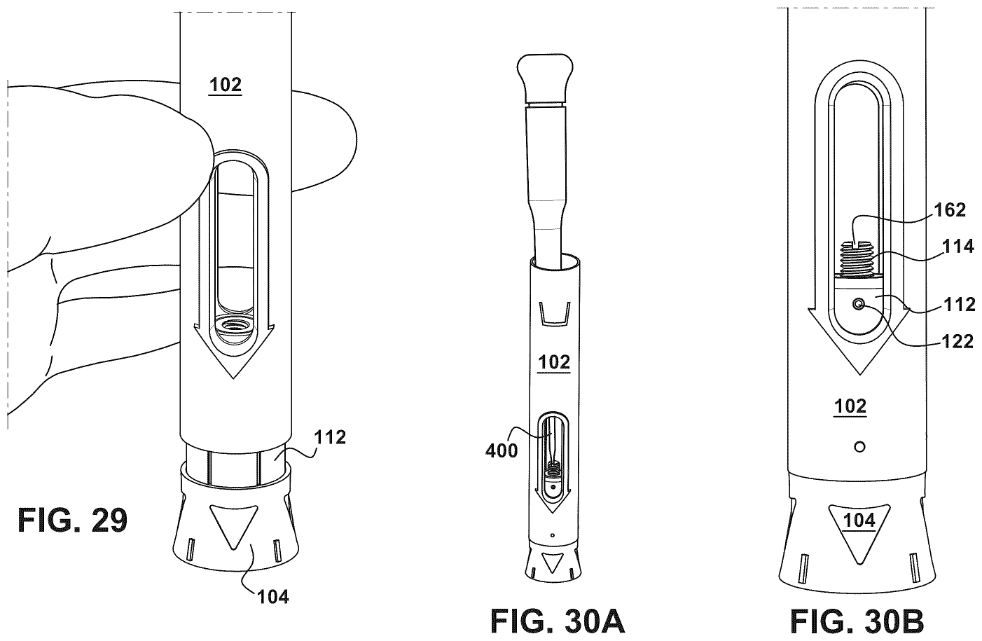

The upper magnet housing 112 may then be installed into the body 102. This installation may optionally be augmented by applying the adhesive 160, e.g., super glue, to a gloved finger (FIG. 28A), and with the finger, applying the adhesive 160 to the inner surface of the body 102 (FIG. 28B). In other embodiments, the use of adhesive 160 may be omitted or deferred as described elsewhere herein. Where the use of adhesive 160 is omitted, the fastener 122 serves to limit translation of the upper magnet housing 112 in use. Regardless of the presence or absence of the adhesive 160, the upper magnet housing 112 may then be installed using the cap assembly (FIG. 29). The upper magnet housing 112 may be pressed up into the body 102, such that about 5 mm of the upper magnet housing 112 extends distally beyond the distal lip of the body 102.

In step 206 (FIG. 7), the upper magnet 120 is assembled into the body 102, and the cap 104 is assembled onto the body 102, thereby forming the pseudo sample 100. The assembled cap 104 may be placed on the upper magnet housing 112, and the upper magnet housing 112 may be pressed proximally into the body 102, until the distal lip of the cap 104 aligns with the body 102. Once the adhesive 148 (applied in step 204 of the method of FIG. 7, and shown in FIG. 18) has cured, e.g. for at least 30 minutes, the cap 104 may be adjusted until it aligns concentrically with the body 102. For example, a visual indicator on the body 102 may be concentrically aligned with a visual indicator on the cap 104. In certain embodiments, the visual indicators may be, e.g., arrows, as shown in FIGS. 30A-30B. A flathead screwdriver 400 may then be used to apply force in a distal direction onto the upper magnet housing 112 to ensure contact between the upper magnet housing 112 and the cap magnet housing 130 without misaligning the cap 104 and the body 102. In particular, the tip of the flathead screwdriver 400 may be positioned within the slit 162 in the proximal end of the rod 114.

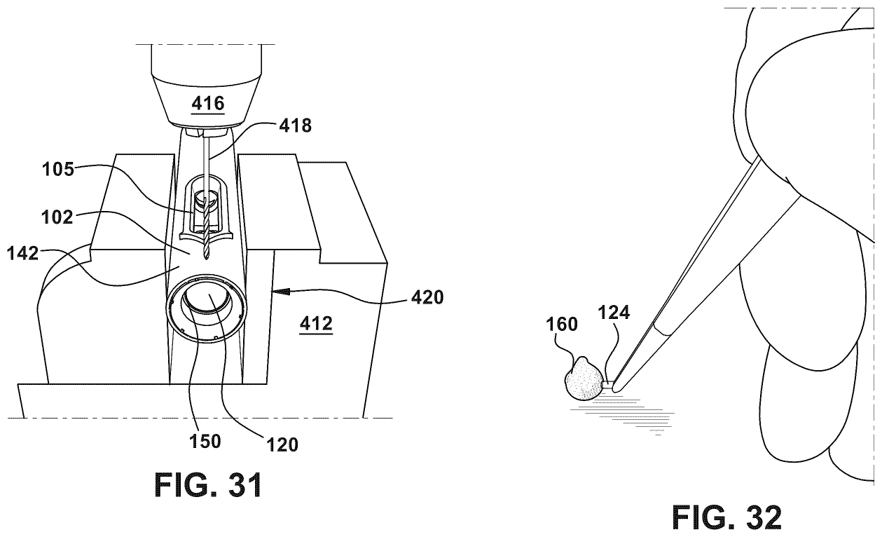

The upper magnet housing 112 may then be rotationally and axially affixed in position within the body 102. This may include drilling one or more bores 128 in the body 102 near a distal end thereof. In various embodiments, one, two, three, or more bores 128 may be drilled as described herein. Each bore 128 may extend radially inward from the outer circumferential surface of the wall 134 of body 102, and be adapted to receive pins 124 therein. The bores 128 may be formed by inserting a drill bit 418, e.g., 0.595 in. (15.113 mm), into the precision drill press chuck 416 and tightening it into place using the chuck key. The body 102 may then be inserted into the precision drill press vise 412, oriented horizontally such that the longitudinal axis of the body 102 is parallel to the v-block 420. The tip of the drill bit 418 may be circumferentially aligned with an indicator or feature on the body 102. For example, as shown in FIG. 31, the drill bit 418 is aligned with a tip of an arrow at the distal end of the window opening 105. The tip of the drill bit 418 may further be axially located along an extent of the upper magnet housing 112 having the larger first outer diameter 142, and between the distal end of the window opening 105, and the distal end of the body 102. In certain embodiments, the bore 128 may be drilled about 5 mm from the distal end of the body 102, and with a diameter of, e.g., about 3.5 mm. A second such bore 128 may be located and drilled at another circumferential location, e.g., circumferentially aligned with a second indicator on the body. In certain embodiments, the second indicator may be a tip of an arrow at the distal end of a second window opening 105 in the body 102, which may be about 180 degrees around the circumference of the body 102. The second bore 128 may similar be axially located distally of the window opening 105, and may be, e.g., about 5 mm from the distal end of the body 102. The drill bit may then be positioned at a third location to drill a third bore 128. The third bore 128 may be circumferentially aligned with an indicator such as, e.g., a mold seam line on the body 102, and may be axially aligned with the other bores 128. The body 102 may be removed from the precision drill press vise 412 once the bores 128 are drilled.

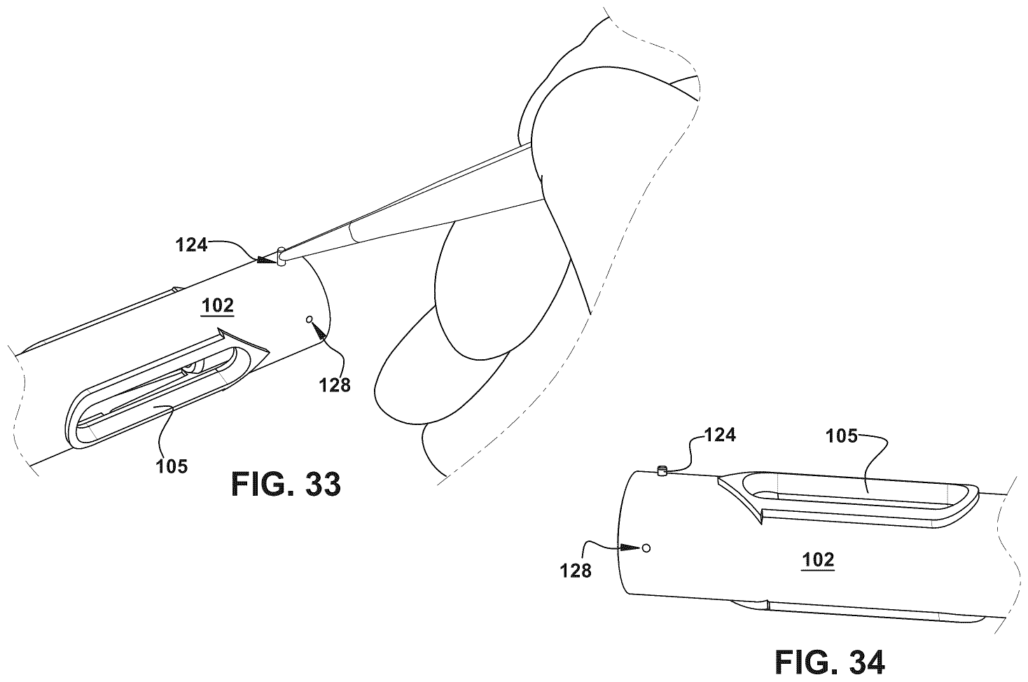

A pin 124 such as, e.g., a dowel pin made of 18-8 stainless steel, may then be inserted into each of the bores 128 to rotationally and axially secure the upper magnet housing 112 to the body 102. The pins 124 may then be affixed within their corresponding bores 128 with an adhesive 160 such as, e.g., super glue. For example, a user may apply the adhesive 160 to a substrate, e.g., a paper towel, and using tweezers, dip a tip of the pin 124 into the adhesive 160 (FIG. 32) prior to inserting the pin 124 into the corresponding bore 128 in the body 102 (FIG. 33). The body 102 may then be placed onto the plate of the arbor press so that the window opening 105 does not contact the plate (FIG. 34). The handwheel on the arbor press may be used to push each pin 124 into its corresponding bore 128 until the pin 124 is flush with the outer circumferential surface of body 102. This process may be repeated for each pin 124.

With reference to the method of FIG. 7, once the pseudo sample 100 is assembled, the method further includes step 207, which includes confirming that the pseudo sample 100 is set to the appropriate cap removal force. The cap removal force of the pseudo sample 100 may be tested using a test frame such as an INSTRON test frame (INSTRON is a trademark of Illinois Tool Works, Norwood, MA). Depending on the outcome of the test, greater or lesser cap removal force may be desired. The pseudo sample may then be adjusted accordingly as described herein. Namely, to reduce cap removal force, the upper magnet 120 may be translated proximally, i.e. away from the cap magnet 132, and to increase cap removal force, the upper magnet 120 may be translated distally, i.e. toward the cap magnet, to bring the magnets closer together. Such adjustments may be made by accessing the fastener 122 via the window opening 305 as described herein above. In other embodiments, a user may alternatively gain access, or gain additional access, by withdrawing the pins 124 from the bores 128, and withdrawing the upper magnet housing 112 from the body 102. The fastener 122 may be withdrawn from its corresponding opening 126, e.g., by inserting a tool through the window opening 305. Where the fastener 122 is a set screw, the tool may be a hex key 464, which may be used to turn the set screw counter clockwise to withdraw the set screw from its corresponding opening 126. With the fastener 122 removed, the adjustable magnet assembly 110 may be adjusted to change the relative height of the upper magnet 120, e.g., by using a flathead screwdriver 400 in slit 162 to turn the assembly in a first direction, e.g. clockwise, to increase the cap removal force, or a second direction, e.g. counter clockwise, to decrease the cap removal force. The fastener 122 may then be replaced and locked, e.g., by turning the hex key 464, e.g. in a clockwise direction.

Placement of the fastener 122 in the absence of adhesive 160 as described relative to FIGS. 28A-28B, may serve to fix the axial position of the upper magnet 120, thereby fixing the cap removal force at the desired amount. In certain embodiments, the pseudo sample 100 may be used to validate a test method as described herein below without further assembly, e.g., of the rear sub-assembly 301, and without adhesive 160 as shown in FIGS. 28A-28B. Such deployment may preserve the ability of the pseudo sample 100 to be adjusted in the future with respect to the desired cap removal force.

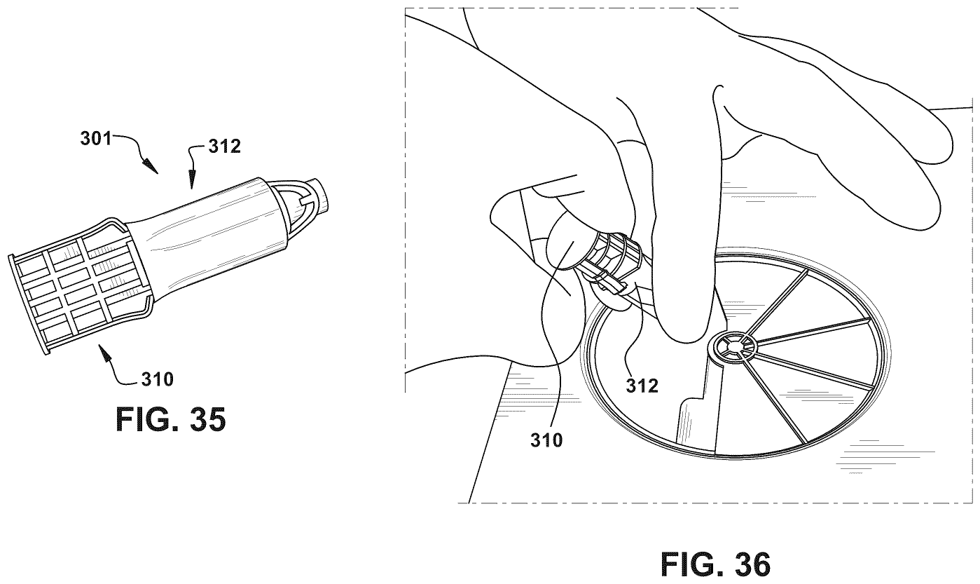

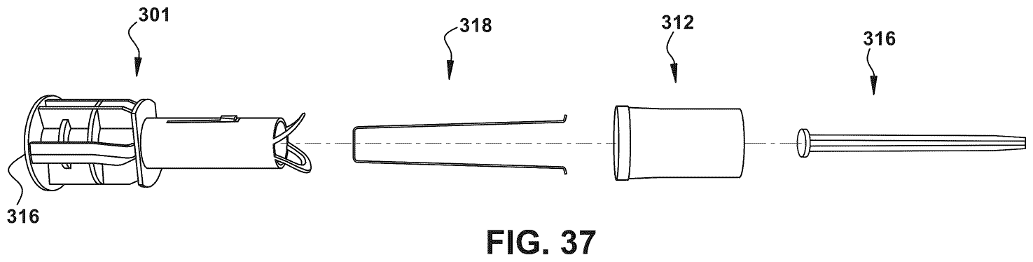

In certain embodiments, once a final adjustment is made, or an adjustment is deemed final, the position of the upper magnet 120 may be further secured using adhesive 160 as described herein above. Following the application of adhesive 160, the pseudo sample 100 may not be further adjustable. The rear sub-assembly 301 may then be adapted from that of the production sample 300 to the pseudo sample 100. This may be achieved by picking up the rear sub-assembly 301 by the end cap 310 without touching the rotator sheath 312 (FIG. 35) to remove a spring from the rear sub-assembly. With the distal end of the rear sub-assembly 301 aimed into a dampened collection receptacle, e.g., at an angle relative thereto, the spring may be released into the dampened collection receptacle by twisting the sheath 312 counter clockwise (FIG. 36). This may include the steps of pulling the spring guide rod 316 and the rotator or sheath 312 off the rear sub-assembly 301, and using a pair of needle nose pliers to remove the U-bracket 318 from the end cap 310 (FIG. 37). The end cap 310 may then be installed back onto the pseudo sample 100. The tabs of the end cap 310 may be aligned with slots at the proximal end of the body 102, and pressure may be applied on the end cap 310 in a distal direction, until the tabs lock into place, giving an auditory indication of locking, e.g., a click.

Although the assembly method 200 is depicted with respect to one particular autoinjector device, it is understood that the methods described herein may apply equally, and be adaptable to other two-step autoinjectors, as well as other needle-based drug delivery devices.

### Methods of Performing Test Method Validations Using the Pseudo Sample

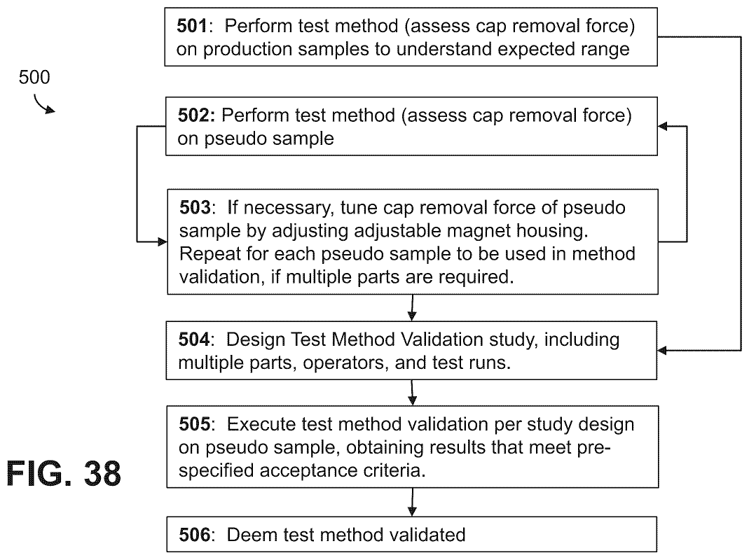

With reference to the flow chart depicted in FIG. 38, a pseudo sample 100 as shown in FIGS. 1-6, or as prepared according to the method of FIG. 7, may be used to validate a test method according to method 500. In particular, the pseudo sample 100 may be used to validate test methods which are themselves used to test the cap removal force for drug delivery devices such as, e.g., autoinjectors, prefilled syringes, and other drug delivery devices. These test methods ensure that the force required to remove the cap from the delivery device is neither too great nor too small. Validation of the test method ensures that the test method employed to assess cap removal force is accurate, repeatable, and reproducible.

The method 500 may include at step 501, performing the test method on one or more production samples to assess and understand the expected range of cap removal forces therefor, using a test frame such as, e.g., an INSTRON test frame. A load cell in communication with the test frame may be adapted to convert a force required to remove the cap from the drug delivery device, into a quantifiable signal representing mechanical displacement. The signal may be transmitted to a computing device, which in turn may output a force in units, e.g., newtons, that is required to remove the cap from the body of the drug delivery device. In various embodiments, the load cell may be, e.g., a strain gauge, a pneumatic load cell, a hydraulic load cell, or a piezoelectric load cell.

Validation of the test method demonstrates that the test method accurately, repeatably, and reproducibly quantifies the force required to de-cap the drug delivery device, without damaging or affecting the drug delivery device. In step 502, the test method may be performed in an identical manner on a pseudo sample 100 as described herein, to assess the cap removal force of the pseudo sample. In order for a pseudo sample to be suitable for use in test method validation, the pseudo sample must allow for non-destructive testing, utilize the same test equipment and fixtures as a production sample, and produce functional outputs within the expected measurement range of a production sample. Additionally, fabrication of the pseudo sample may be documented as part of the validation protocol or through a separate build document.

At step 503, if necessary, the cap removal force of the pseudo sample may be tuned or adjusted, e.g. by adjusting a relative height of the upper magnet 120 within the upper magnet housing 112, such that the cap removal force of the pseudo sample falls within a pre-determined range suitable for the production sample. If the cap removal force at step 502 exceeds an upper bound of the pre-determined range, the relative height of the upper magnet 120 may be increased in step 503, i.e., the upper magnet 120 may be translated proximally, thereby increasing the distance between the upper magnet 120 and the cap magnet 130, and reducing the cap removal force. If the cap removal force at step 502 is below a lower bound of the pre-determined range, the relative height of the upper magnet 120 may be decreased in step 503, i.e., the upper magnet may be translated distally, thereby decreasing the distance between the upper magnet 120 and the cap magnet 130, and increasing the cap removal force. Steps 502 and 503 may be performed iteratively as desired for each pseudo sample to be used in the test method validation, if more than one are required. When the cap removal force is determined to be within the pre-determined range in step 502 for each pseudo sample, step 503 may be bypassed, and the TMV process may advance to step 504. In certain embodiments, a plurality of pseudo samples may be used, representing a range of in-specification values, e.g., nominal cap removal forces, and a range of out of specification values, e.g., samples to challenge the method.

In step 504, a test method validation study is designed, including multiple parts, operators, and test runs, to demonstrate that accuracy and precision are not affected by the test method throughout the range of possible cap removal values. In certain embodiments, the test method validation study may also demonstrate the behavior of the test method outside of the pre-determined measurement range, e.g., above 35 N or below 4 N.

In step 505, the test method validation is executed according to the study design of step 504, obtaining results that meet pre-specified acceptance criteria.

The test method may be deemed to be validated in step 506 upon the completion of the plurality of test runs in step 505, which may include multiple test runs of multiple “tuned” pseudo samples that cover a range of cap removal force values, including low, medium, and high, test runs performed by multiple operators. In certain embodiments, the plurality of test runs may be, e.g., at least five.

### EMBODIMENTS

Embodiments of the present disclosure may include the following features:

Item 1. A pseudo sample configured for validating a test method, the test method comprising measuring a force required to remove a cap from a drug delivery device, the pseudo sample comprising: an upper magnet movably secured within a body of the drug delivery device; and a cap magnet secured within the cap, wherein an attraction between the upper magnet and the cap magnet is configured to deliver a force corresponding to the force required to remove the cap from the body of the drug delivery device.

Item 2. The pseudo sample of item 1, further comprising: an upper magnet housing disposed about the upper magnet and secured within the body of the drug delivery device.

Item 3. The pseudo sample of item 2, further comprising: an adjustable magnet assembly movably affixed within the upper magnet housing, and affixed to the upper magnet, wherein the adjustable magnet assembly is adapted to translate the upper magnet proximally or distally relative to the upper magnet housing and the body of the drug delivery device.

Item 4. The pseudo sample of item 3, wherein the adjustable magnet assembly further comprises: the upper magnet, wherein the upper magnet is enclosed in a case, the case including a stud extending proximally therefrom; a coupler sleeve disposed over a proximal end of the stud; and a rod, wherein a distal end of the rod is disposed within the coupler sleeve.

Item 5. The pseudo sample of item 4, wherein the rod and the stud each comprise external threads, and the coupler sleeve comprises internal threads adapted to engage the external threads of the rod and stud.

Item 6. The pseudo sample of item 5, further comprising a heat-set threaded insert disposed about a portion of the rod that is proximal of the coupler and within the upper magnet housing, wherein the heat-set threaded insert comprises internal threads adapted to engage the external threads of the rod.

Item 7. The pseudo sample of item 6, further comprising: an opening extending through a thickness of a wall of the upper magnet housing and through a thickness of the heat-set threaded insert; and a fastener disposed in the opening, the fastener adapted to maintain a position of the adjustable magnet assembly relative to the upper magnet housing.

Item 8. The pseudo sample of item 4, wherein the rod comprises steel.

Item 9. The pseudo sample of item 2, further comprising: a bore extending through a full thickness of a wall of the body of the drug delivery device, and through a partial thickness of a wall of the upper magnet housing; and a pin disposed within the bore, thereby affixing an axial position of the upper magnet housing relative to the body of the drug delivery device.

Item 10. The pseudo sample of item 2, wherein the upper magnet housing comprises: a first outer diameter at a distal end thereof; and a second outer diameter at a proximal end thereof, the second outer diameter being smaller than the first outer diameter; and a shoulder disposed at a transition between the first and second outer diameters, wherein the shoulder is adapted to engage a feature on an inner surface of a wall of the body of the drug delivery device, thereby limiting proximal translation of the upper magnet housing relative to the body of the drug delivery device.

Item 11. The pseudo sample of item 1, further comprising a cap magnet housing disposed about the cap magnet and within the cap, wherein the cap magnet housing is axially affixed to each of the cap magnet and the cap.

Item 12. The pseudo sample of item 1, wherein the cap magnet comprises neodymium.

Item 13. The pseudo sample of item 1, wherein the upper magnet housing and the cap magnet housing are formed by additive manufacturing.

Item 14. The pseudo sample of item 1, wherein the drug delivery device is an autoinjector.

Item 15. The pseudo sample of item 1, wherein the drug delivery device is a pre-filled syringe.

Item 16. The pseudo sample of item 1, wherein the standard cap removal force is repeatable and reproducible.

Item 17. A method of assembling a pseudo sample configured for validating a test method, the test method comprising measuring a force required to remove a cap from a drug delivery device, the method comprising: disassembling a portion of a body of a drug delivery device corresponding to the drug delivery device being subjected to the test method; disassembling a portion of a cap of the drug delivery device corresponding to the drug delivery device being subjected to the test method; assembling the cap of the pseudo sample, including affixing a first magnet therein; assembling the body of the pseudo sample, including affixing a second magnet therein, wherein the second magnet is adapted to engage the first magnet when the cap is disposed on the body of the pseudo sample; and confirming that the engagement of the first and second magnets generates a desired cap removal force.

Item 18. The method of item 17, wherein the second magnet is axially adjustable with respect to the body of the pseudo sample, and wherein adjustment of the second magnet in a proximal direction reduces the cap removal force, and adjustment of the second magnet in a distal direction increases the cap removal force.

Item 19. A method for validating a test method, the test method comprising measuring a force required to remove a cap from a drug delivery device, and the method comprising: performing the test method on the drug delivery device; and performing the test method on a pseudo sample as claimed in claim 1 a plurality of times; and confirming that a substantially same result is obtained in every performing step.

Item 20. The method of item 19, further comprising: adjusting the pseudo sample to increase or decrease the force required to remove the cap therefrom, such that the force required to remove the cap from the pseudo sample is substantially the same as the force required to remove the cap from the drug delivery device.

What is claimed is:

1. A pseudo sample configured for validating a test method, the test method comprising measuring a force required to remove a cap from a drug delivery device, the pseudo sample comprising:
an upper magnet adjustably fixed within a body of the drug delivery device; and
a cap magnet secured within the cap,
wherein an attraction between the upper magnet and the cap magnet is configured to deliver a force corresponding to the force required to remove the cap from the body of the drug delivery device.

2. The pseudo sample of claim 1, further comprising:
an adjustable magnet assembly including the upper magnet, the adjustable magnet assembly being disposed within the body of the drug delivery device, and adapted to transport the upper magnet in a distal or proximal direction within the body of the drug delivery device, and to fix a position of the upper magnet after transport.

3. The pseudo sample of claim 2, wherein the adjustable magnet assembly comprises:
an upper magnet housing disposed about the upper magnet and within the body of the drug delivery device,
wherein the adjustable magnet assembly is adapted to translate the upper magnet proximally or distally relative to the upper magnet housing.

4. The pseudo sample of claim 3, wherein the adjustable magnet assembly further comprises:
a case at least partially enclosing the upper magnet, and including a stud extending proximally therefrom;
a coupler sleeve disposed over a proximal end of the stud; and
a rod having a distal end thereof disposed within the coupler sleeve,
wherein the case, the coupler sleeve, and the rod are disposed within the upper magnet housing.

5. The pseudo sample of claim 4, wherein the rod and the stud each comprise external threads, and the coupler sleeve comprises internal threads adapted to engage the external threads of the rod and stud.

6. The pseudo sample of claim 5, further comprising a heat-set threaded insert disposed about a portion of the rod that extends proximally beyond the coupler sleeve and within the upper magnet housing,
wherein the heat-set threaded insert comprises internal threads adapted to engage the external threads of the rod.

7. The pseudo sample of claim 6, further comprising:
an opening extending through a thickness of a wall of the upper magnet housing and through a thickness of the heat-set threaded insert; and
a fastener disposed in the opening, and adapted to maintain a position of the adjustable magnet assembly relative to the upper magnet housing.

8. The pseudo sample of claim 4, wherein the rod comprises steel.

9. The pseudo sample of claim 2, further comprising:
a bore extending through a full thickness of a wall of the body of the drug delivery device, and through a partial thickness of a wall of the upper magnet housing; and
a pin disposed within the bore, thereby affixing an axial position and a rotational position of the upper magnet housing relative to the body of the drug delivery device.

10. The pseudo sample of claim 2, wherein the upper magnet housing comprises:
a first outer diameter at a distal end thereof; and
a second outer diameter at a proximal end thereof, the second outer diameter being smaller than the first outer diameter; and
a shoulder disposed at a transition between the first and second outer diameters, wherein the shoulder is adapted to engage a feature on an inner surface of a wall of the body of the drug delivery device, thereby limiting proximal translation of the upper magnet housing relative to the body of the drug delivery device.

11. The pseudo sample of claim 1, further comprising a cap magnet housing disposed about the cap magnet and within the cap, wherein the cap magnet housing is axially affixed to each of the cap magnet and the cap.

12. The pseudo sample of claim 1, wherein one or more of the cap magnet or the upper magnet comprises neodymium.

13. The pseudo sample of claim 1, wherein one or more of the upper magnet housing or the cap magnet housing are formed by additive manufacturing.

14. The pseudo sample of claim 1, wherein the drug delivery device is an autoinjector.

15. The pseudo sample of claim 1, wherein the drug delivery device is a pre-filled syringe.

16. The pseudo sample of claim 1, wherein the standard cap removal force is repeatable and reproducible.

17. A method of assembling a pseudo sample configured for validating a test method, the test method comprising measuring a force required to remove a cap from a drug delivery device, the method comprising:
providing a drug delivery device corresponding to the drug delivery device to be subjected to the test method;
disassembling a portion of a body of the drug delivery device;
disassembling a portion of a cap of the drug delivery device;
assembling a first magnet into the cap;
assembling a second magnet into the body,
assembling the cap onto the body to form the pseudo sample, such that the second magnet is adapted to magnetically engage the first magnet; and
confirming that the magnetic engagement of the first and second magnets generates a desired cap removal force.

18. The method of claim 17, wherein the second magnet is axially adjustable with respect to the body of the pseudo sample, and
wherein adjustment of the second magnet in a proximal direction reduces the cap removal force, and adjustment of the second magnet in a distal direction increases the cap removal force.

19. A method for validating a test method, the test method comprising measuring a force required to remove a cap from a drug delivery device, and the method comprising:
performing the test method on the drug delivery device; and
performing the test method on a pseudo sample as claimed in claim 1;
designing a study to validate the test method, wherein the study includes a plurality of test runs;
executing the study to validate the test method; and
validating the test method upon obtaining results during the executing that meet pre-specified acceptance criteria.

20. The method of claim 19, further comprising:
after performing the test method on the pseudo sample, adjusting the pseudo sample to increase or decrease the force required to remove the cap therefrom, and
repeating the test method on the adjusted pseudo sample.
